(19) United States
(12) Patent Application Publication
(10) Pub. No.: US 2024/0216326 A1
(43) Pub. Date: Jul. 4, 2024

(54) TOPICAL FORMULATIONS COMPRISING BENZOYL PEROXIDE AND AZELAIC ACID, AND USE THEREOF

(71) Applicant: NOON AESTHETICS M.R. LTD., Tel Aviv (IL)

(72) Inventors: Masha Minkin, Tel Aviv (IL); Eran Rosman, Tel Aviv (IL)

(21) Appl. No.: 18/557,089

(22) PCT Filed: Apr. 30, 2022
(86) PCT No.: PCT/IB2022/054017

§ 371 Date: Oct. 25, 2023

Related U.S. Application Data
(60) Provisional application No. 63/182,196, filed on Apr. 30, 2021.

(51) Int. Cl. A61K 31/327; A61K 9/16; A61K 31/10; A61K 31/19; A61K 31/20; A61K 33/14; A61K 47/10; A61P 17/10

(52) U.S. Cl. CPC A61K 31/327; A61K 9/16; A61K 31/10; A61K 31/19; A61K 31/20; A61K 33/14; A61K 47/10; A61P 17/10

(57) ABSTRACT

Compositions comprising benzoyl peroxide, azelaic acid, a strontium salt, and methylsulfonylmethane (MSM) are provided, formulated as one or more dosage forms, wherein at least one dosage form comprises both benzoyl peroxide and azelaic acid. These compositions are formulated as cosmetic or medicinal products useful in treating skin diseases, disorders or conditions which may benefit form topical co-administration of BPO and azelaic acid. These skin care products provide dermal shielding against neurogenic inflammation, stinging, itching, burning, redness, irritation, and/or other sensations and feelings associated with topical application of BPO and/or azelaic acid.

### FIELD OF THE INVENTION

The current disclosure relates to formulations combining benzoyl peroxide, azelaic acid, strontium and methylsulfonylmethane (MSM) for treatment of certain dermatological diseases and conditions.

### BACKGROUND

Various skin diseases and conditions are optimally treated by topical application of more than one active agent. For example, acne vulgaris and rosacea are multi-factorial dermatologic diseases that are treated by several drugs or active agents. Separate applications of several drugs, sometimes provided sequentially within limited time intervals, often present a certain discomfort, burden and/or encumbrance to the patients. Moreover, often, a combination of two or more active agents, enhances or intensifies undesired side effects which would otherwise be lower if each of the active agents is administered separately. Thus, a patient's inability to tolerate and withstand such side effects outweighs the advantages and benefits of a combination therapy.

Benzoyl peroxide (BPO) is a well-known ingredient for fighting acne. Available in over-the-counter (OTC) gels, cleansers, and spot treatments, this ingredient comes in different concentrations for mild to moderate breakouts. High percentage benzoyl peroxide products contain up to 10% (w/w) BPO, facial products usually contain up to 4% BPO. Benzoyl peroxide works to treat and prevent acne by killing bacteria underneath the skin, as well as helping the pores shed dead skin cells and excess sebum (oil). While considered safe for most people, benzoyl peroxide can cause serious side effects such as dryness, redness, excessive peeling, itching and general irritation at the site of application. People with sensitive skin cannot use BPO.

Azelaic acid is a naturally occurring acid found in grains such as barley, wheat, and rye. It has antimicrobial and anti-inflammatory properties, which make it effective in the treatment of skin conditions like acne and rosacea. Azelaic acid is available in gel, foam, and cream form as prescribed topical preparations, containing 15% (w/w) or more of azelaic acid. Some over-the-counter products contain smaller amounts. Azelaic acid works by clearing pores of bacteria that may be causing irritation or breakouts, reducing inflammation so acne becomes less visible, less red, and less irritated, and encouraging cell turnover so the skin heals more quickly, and scarring is minimized.

The acid has some side effects, such as skin burning or tingling, dryness, redness and peeling at the site of application. Since it works slowly, azelaic acid is often prescribed along with other forms of acne treatment. Some studies report that azelaic acid cream may be as effective as benzoyl peroxide and tretinoin (Retin-A) for the treatment of acne.

There is no documentation for use of both BPO and azelaic acid as combined therapy for treating skin pathologies.

There is still an unmet need for innovative topical formulations and skin care products combining two or more active agents, such as BPO and azelaic acid, wherein combination of which may be advantageous in certain skin pathologies but are not co-applied in current practice due to the substantial and extensive accumulated skin damage which might be facilitated due to their co-application.

### SUMMARY

Benzoyl peroxide (BPO) and azelaic acid, two commonly used anti-acne active agents, are also known to cause various adverse side effects when applied each alone, more so when applied together in any combination. The present disclosure is based on a discovery by the present inventors that BPO and azelaic acid may, nevertheless, be applied as a single unit dose form when provided in combination with strontium and/or methylsulfonylmethane (MSM). Moreover, such combination was proven to be highly effective in reducing, preventing and/or eliminating the development, incidence and severity of neurogenic inflammation commonly associated with topical application of benzoyl peroxide and azelaic acid in cosmetic products and/or medicinal products such as anti-acne and/or anti-rosacea products.

In one aspect, the present disclosure relates to a composition comprising benzoyl peroxide, azelaic acid, a strontium salt, MSM and a dermatologically acceptable carrier, wherein the composition is formulated as one or more dosage forms, such that at least one dosage form comprises both benzoyl peroxide and azelaic acid.

In the compositions disclosed herein, the concentration of benzoyl peroxide may be from 0.10% to 10% w/w, and the concentration of azelaic acid may be from 0.1% to 20% w/w.

The strontium salt may be strontium chloride, strontium acetate, strontium nitrate or strontium chloride hexahydrate.

A contemplated composition may further comprise additional active ingredient such as, but not limited to, an alpha hydroxy acid (AHA), a beta hydroxy acid (BHA), a retinoid, an alpha keto acid, a dicarboxylic acid, arbutin, resorcinol, hydroquinone, kojic acid, myristic acid, sodium laureth sulfate, disodium laureth sulfosuccinate, sulfur, vitamin C, a vitamin C derivative, a cannabinoid, an azelaic acid derivative, salt and/or a prodrug, diaryl peroxide, alkyl aryl peroxide, and/or a cycloalkyl aryl peroxide.

Any of the compositions disclosed herein may be formulated or fabricated as a cosmetic or skin care product, or a medicament (medicinal product) for topical application, using the appropriate excipients, carriers, penetration enhancers and the like. None-limiting examples of skin care/medicinal formulations or products contemplated herein include ointment, cream, spread, lotion, an oil, solution, emulsion, nano-emulsion, gel, paste, milk, aerosol, powder, or foam.

The cosmetic or medicinal products are physically and/or chemically stable for a period of at least 3 months under 4 to 40° C.

In another aspect, the present disclosure relates to methods of treatment employing any of the compositions and/or a cosmetic product or medicament disclosed herein.

In some embodiments, a contemplated method is useful for treating, preventing, ameliorating and/or relieving a skin disease, disorder or conditions which may benefit form topical co-administration of BPO and azelaic acid. Skin disease, disorder or condition, which may be treated include, but are not limited to, acne, rosacea or seborrhea.

In some embodiments, a contemplated method is useful for treating, preventing, ameliorating, relieving and/or reducing neurogenic inflammation.

In some embodiments, a contemplated method is useful for treating, preventing, ameliorating, relieving and/or reducing one or more of: stinging, itching, burning, redness, irritation, and/or other sensations and feelings associated with topical application of BPO and azelaic acid.

### DETAILED DESCRIPTION

The present disclosure relates to the surprising discovery by the present inventors that simultaneous topical application of benzoyl peroxide and azelaic acid, each alone or in any combination thereof, at concentrations prevailing in common dermatologic products, combined with application of a strontium salt and/or MSM, substantially reduced serious side effects such as irritation, itching, erythema, burning, and the like, due to topical application of benzoyl peroxide and/or azelaic acid. The calming, irritation reducing effect of co-application of strontium and MSM on the topically applied combination of benzoyl peroxide and azelaic acid was immediate, effective and for a long period prevented the sensations of stinging, itching, burning, erythema and other sensations and feelings associated with neurogenic inflammation.

It has been, thus, envisioned by the present inventors that co-application of two or more active agents for treating various dermatologic diseases and conditions, which has been avoided thus far due to serious side effects to the skin, may now be enabled if such co-application is combined with application of strontium and/or MSM.

There is still a need for innovative topical formulations combining two or more active agents, which in current practice are not combined, or formulated as a single product, e.g., a cream or ointment, that can be delivered to subjects in need thereof as a stable composition featuring excellent cosmetic and/or therapeutic properties and minimum undesired side effects. This need is yet unmet for combinations of certain active agents that although they provide enhanced and improved therapeutic/cosmetic outcomes when combined are nevertheless not combined due to accumulative intolerable side effects.

Benzoyl peroxide (molecular formula: C₁₄H₁₀O₄; MW: 242.23 g/mol), is an organic peroxide having a structural formula (C₆H₅—C(═O)O—)₂ (often abbreviated as (BzO)₂ or BPO), available as a white granular crystalline solid with a faint odor of benzaldehyde. This peroxide presents antibacterial, irritant, keratolytic, comedolytic, and/or anti-inflammatory activities. As a medication, benzoyl peroxide is mostly used to treat acne, either alone or in combination with other treatments. Benzoyl peroxide works to treat and prevent acne by killing bacteria underneath the skin, as well as helping the pores shed dead skin cells and excess sebum (oil). This anti-acne active agent works particularly well for inflammatory acne such as acne vulgaris, which is characterized by red bumps that contain pus (pustules, papules, cysts, and nodules) instead of whiteheads and blackheads. Due to its irritant effect, benzoyl peroxide increases turnover rate of epithelial cells, thereby peeling the skin and promoting the resolution of comedones. It has also been suggested that BPO improves retention hyperkeratosis of infundibular hair follicles.

Topical medications containing 2.5-10% BPO for treatment of, e.g., acne vulgaris and/or rosacea, are widely used in Asian countries like Korea, Singapore and Hong Kong, as well as Europe and the USA, where the medical guidelines recognize BPO as a standard of care for acne vulgaris. However, application of these products on the skin often results in intolerable side effects such as burning, stinging, irritation, inflammatory skin, peeling, and/or redness at treatment sites. Often, due to high levels of these side effects, the use of such products has only a modest efficacy, and patients often cannot complete their treatment regimens.

Developing a BPO product with an improved efficacy and tolerability has thus been challenged. Sol-gel technology has been used to develop a microencapsulated formulation of BPO (E-BPO) for the treatment of rosacea that may provide some relief to the serious side effects caused by the free active agent. Commercially available is the product E-BPO Cream, 5% formulation, comprising BPO encapsulated within a porous silica shell. The capsules form a barrier between the skin and the BPO crystals or other ingredients, allowing for the active drug to be released in a timely fashion (sustained release), resulting in less skin irritation and a much higher tolerability and amenability of the medication in patients. However, the therapeutic effect of encapsulated BPO is very slow. For example, improvement of rosacea symptoms progresses slowly over several months (e.g., over 40-52 weeks).

Azelaic acid, a dihydroxy acid (HOOC(CH₂)₇COOH), is a naturally occurring saturated dicarboxylic acid which, on topical application (usually as a 20% cream), has been shown to be effective in the treatment of comedonal acne and inflammatory (papulopustular, nodular and nodulocystic) acne, rosacea, as well as various cutaneous hyperpigmentary disorders characterized by hyperactive/abnormal melanocyte function such as melasma and lentigo maligna. In addition, azelaic acid has an antiproliferative and cytotoxic effect on the human malignant melanocyte, and it may arrest the progression of cutaneous malignant melanoma. Azelaic acid's anti-inflammatory effect for acne and it's anti-pigment effect is attributed to its ability to block tyrosinase. In controlled studies, topical azelaic acid demonstrated comparable anti-acne efficacy to topical tretinoin, benzoyl peroxide, erythromycin and oral tetracycline, while in patients with melasma, azelaic acid proved at least as effective as topical hydroquinone. Products for treatment of, e.g., acne vulgaris and/or rosacea, comprising azelaic acid in concentrations of 15-25% (w/w) are known.

Azelaic acid may be used alone or paired with other soothing and brightening ingredients like niacinamide, hydroxy acids or antioxidants. It is often paired with hyaluronic acid to provide moisture and/or with a moisturizer and a gentle cleanser or sulfur wash for treating acne or rosacea. Combining azelaic acid with other ingredients is usually highly recommended because azelaic acid is a very stable molecule and because combination therapy may be much more effective than azelaic acid monotherapy.

Well known azelaic acid products include, for example, facial serum and acid boosters. Facial serum is mostly applied to treat pigmentation, and often comprises a combination of azelaic acid with several other potent active ingredients such as retinol, vitamin C and/or Morus alba extract. Acid boosters deliver at least 10% azelaic acid, sometimes combined with salicylic acid, to brighten dark spots and even skin tone. Further products include gels such as gels for acne treatment and/or for skin brightening, often combining azelaic acid (e.g., 2% w/w), niacinamide, salicylic acid and/or hyaluronic acid; gel masks for calming the skin and sooth redness, containing azelaic acid, green tea extract and aloe leaf juice to absorb excess oil without stripping the skin of moisture.

Azelaic acid is generally not well-tolerated and subjects with sensitive skin may experience mild irritation and redness. In some cases, use of azelaic acid products is associated with stronger side effects such as burning, stinging, and/or irritation of medium levels.

Although both BPO and azelaic acid are well known for their therapeutic effects in treating skin diseases and conditions such as acne, rosacea, seborrhea and the like, actual and successful attempts to combine both active agents together, in a single topical application formulation have not been documented. Canadian Patent No. 2,362,343 discloses a method of treating acne vulgaris with two topical compositions a first composition containing benzoyl peroxide and a second composition containing azelaic acid. These compositions demonstrated some improved efficacy over the state-of-the-art compositions known at that time, however, they were not simultaneously topically applied but rather serially, assumingly in order to avoid the serious outcome of the overall side effects exerted by both benzoyl peroxide and azelaic acid.

Combination of strontium with MSM in topical formulations has been previously shown by the present inventors to be useful in reducing the development, incidence and severity of irritation and erythema associated with topically applied skin irritants contained, e.g., in skin treatment products (International Application Publication No. WO 2019/198067).

Strontium salts and MSM are known to rapidly suppress acute sensory irritation (e.g., stinging, burning, pain and/or itching) resulting, e.g., from neurological inflammation, chemical irritants, environmental irritants, allergies, and diseases. It has been theorized that strontium's anti-irritant activity may be related to its ability to selectively suppress activation of Type C Nociceptors (TCN), the only sensory nerves that produce and transmit stinging, burning, pain, and itching sensations.

It is demonstrated herein that benzoyl peroxide and azelaic acid, which are known to cause various adverse side effects when applied each alone, more so when applied together in any combination, may nevertheless be applied as a single unit dose form in the context of various topical products, when provided in combination with strontium, e.g., in the form of a strontium salt, and/or MSM. It is further demonstrated herein that such a combination is useful in reducing, preventing and/or eliminating the development, incidence and severity of neurogenic inflammation commonly associated with topical application of benzoyl peroxide and azelaic acid in cosmetic products and/or medicinal products such as anti-acne and/or anti-rosacea products.

Surprisingly, a synergistic effect was obtained by combining the two materials, BPO and azelaic acid, in the presence of strontium salt and MSM. Namely, a higher efficacy: stronger effects and more significant results, was observed than the efficacy predicted by the simple sum of the effects exerted by each of BPO and azelaic acid applied alone. As it may be desirable to use small amounts of BPO and/or azelaic acid, the synergistic effect may enable the use of effective combinations of small amounts of these active agents, which otherwise may not be as effective.

Neurogenic inflammation involves a change in function of sensory neurons due to inflammatory mediators. Neurogenic inflammation induces an enhanced local release of neuropeptides such as substance P, calcitonin gene-related peptide (CGRP), neurokinin A (NKA), and endothelin-3 (ET-3) from the sensory nerve endings. Release of these inflammatory mediators is thought to be triggered by the activation of ion channels that are the principal detectors of noxious environmental stimuli.

Neurogenic inflammation may be caused, for example, by one or more of: skin diseases, disorders and conditions, allergic reactions, reaction to topical skin irritants, drug application, chemicals, temperature change, eczema, environmental exposure, bacterial, fungal, viral, or parasitic infections, change in pH, beauty and cleansing products, laser and other light-based treatments, radio frequency (RF) and ultrasound treatments, bites, or poisonous plants. Neurogenic inflammation may cause local pain, irritation, stinging, burning, itching, edema, erythema, unpleasant sensations and other side effects.

Trials conducted by the present inventors demonstrated that when a combination of a strontium salt and MSM was co-applied topically along with a combination of BPO and azelaic acid, it immediately and effectively prevented development of neurologic inflammatory response and symptoms related thereto as defined herein, and other sensations and feelings associated with skin irritant products containing BPO or azelaic acid.

### Compositions and Formulations

In one aspect, the present disclosure relates to a composition comprising benzoyl peroxide, azelaic acid, strontium, methylsulfonylmethane (MSM) and a dermatologically acceptable carrier, wherein the composition is formulated as one or more dosage forms, wherein at least one dosage form comprises both benzoyl peroxide and azelaic acid.

The term “dosage form”, as used in the context of the present disclosure, sometimes also interchangeably with the term “unit dose”, refers to a pharmaceutical and/or cosmetic composition in a form in which it is intended to be used (applied, administered) and/or marketed, comprising a specific mixture of active ingredients and, optionally, inactive components (excipients), apportioned into a particular dose. Depending on the context, in some embodiments, the term dosage form may sometimes refer not only to the formulation of a pharmaceutical/cosmetic composition's constituent active substance(s) and any blends involved, but also to the way or form it is ultimately configured as a consumable product. In such broader interpretation, and depending on the method/route of administration, dosage forms may be of several types, including many kinds of liquid, solid, and semisolid dosage forms. Common dosage forms include, but are not limited to, pill, tablet, capsule drink or syrup, and the like. For example, a liquid dosage form is the liquid form of a dose of a chemical compound or mixture of compounds used as a drug, medication and/or cosmetic product intended for administration or consumption. Notably, the route of administration (ROA) is dependent on the dosage form of the substance in question. Various dosage forms may exist for a single particular drug and/or cosmetic composition, since different conditions can warrant different routes of administration.

A disclosed composition may be formulated as a single dosage form comprising BPO, azelaic acid, strontium and MSM. Alternatively, a disclosed composition may be formulated as two or more dosage forms. For example, the composition may comprise two dosage forms, a first dosage form comprising BPO and azelaic acid, and a second dosage form comprising strontium, e.g., in the form of a salt, and MSM. A disclosed composition may be formulated, for example, as three dosage forms, wherein a first dosage form comprises BPO and azelaic acid, a second dosage form comprises strontium, e.g., in the form of a salt, and a third dosage form comprises MSM.

Contemplated compositions may comprise azelaic acid as well as various pharmaceutically acceptable derivatives, salts and prodrugs of azelaic acid, such as, but not limited to, sodium or potassium salt of azelaic acid, azeloglycine and/or lower alkyl ester, i.e., C1 to C6, alkyl ester, e.g., methyl azelate. Any one or more of these derivatives, salts and/or prodrugs may be used in addition to, or in place of azelaic acid. Likewise, various pharmaceutically acceptable derivatives, salts and prodrugs of BPO may be used in a contemplated composition, for example, hydrous benzoyl peroxide. Also, in some embodiments, various forms of other peroxides may be used in place of, or in addition to, BPO such as, but not limited to, a diaryl peroxide, alkyl aryl peroxide, and/or a cycloalkyl aryl peroxide, for example, lauroyl benzoyl peroxide and/or cyclohexyl carbanolyl benzoyl peroxide.

Azelaic acid and/or its pharmaceutically acceptable salts, derivative or prodrugs (e.g., azeloglycine, sodium salt of azelaic acid or lower alkyl ester of azelaic acid), may be applied in an amount sufficient to provide from about 0.1 to about 40 weight percent of total composition (w/w), for example, form about 0.1% to about 2% w/w, form about 1% to about 5% w/w, form about 4% to about 8% w/w, form about 5% to about 10% w/w, form about 10% to about 20% w/w, form about 15% to about 30% w/w, form about 22% to about 28% w/w, form about 25% to about 30% w/w, from about 10% to about 30% w/w, form about 25% to about 35% w/w, form about 25% to about 40% w/w, or form about 30% to about 40% w/w, and any subranges and individual values therebetween.

In some embodiments, a contemplated composition comprises azelaic acid in the amount of form about 0.5% to about 25% w/w, form about 5% to about 30% w/w, form about 10% to about 25% w/w, form about 15% to about 35% w/w, about 15% w/w or about 25% w/w.

Benzoyl peroxide, and/or any pharmaceutically acceptable derivative, salt, or substitute peroxide may be present in a total amount sufficient to provide from about 0.1 to about 30 weight percent of total composition (w/w), for example, form about 0.1% to about 2% w/w, form about 1% to about 5% w/w, from about 4% to about 8% w/w, form about 5% to about 10% w/w, from about 8% to about 12% w/w, form about 10% to about 15% w/w, form about 12% to about 20% w/w, form about 15% to about 22% w/w, or form about 20% to about 30% w/w, and any subranges and individual values therebetween.

In some embodiments, a contemplated composition comprises BPO in the amount of form about 2.5% to about 10% w/w, form about 5% to about 10% w/w, form about 8% to about 15% w/w, about 5% w/w or about 10% w/w.

In some embodiments, a contemplated composition may comprise micronized PBO particles (diameter of <10 μm) and micronized azelaic acid particles in a nano-emulsion dosage form.

Micronization is the process of reducing the average diameter of a solid material's particles to few micrometers. An active pharmaceutical ingredient (API) is said to be micronized when its particle size is generally less than 50 microns, which is about 4 to 10 times smaller than conventional drug particles. Micronization is applied in order to improve a drug's bioavailability, aqueous solubility and/or permeability through the body's membranes.

In some embodiments, benzoyl peroxide and/or azelaic acid are present as homogeneously distributed micronized particles in a contemplated composition.

The terms “nano-emulsion” and “micro-emulsion” as used herein, refer to emulsions with nano-size range and micro-sized range particles or droplets, respectively.

Nano-emulsions are thermodynamically stable transparent or translucent colloidal dispersion form of two immiscible liquids, e.g., oil in water (o/w) or water in oil (w/o), stabilized by an interfacial film of surfactant and cosurfactant molecule and having the droplet size 10-100 nm. Advantages of nano-emulsions include increased drug loading, and enhanced bioavailability, good stability, rapid digestibility, protection against degradation, and controlled release. The oily phase can be formulated using different types of lipids and oils such as triglycerides and essential oils to produce nano-emulsions of different physicochemical and biological properties. The aqueous portion may be manipulated by adding different water-soluble components.

Creams, liquids, sprays, and foams are some examples of dosage form in which nano-emulsified BPO and/or azelaic acid can be molded. Nano-emulsion can be formulated with variety of techniques like high-pressure homogenization, ultrasonication, microfluidization, and titrimetric method.

In some embodiments, BPO is encapsuled, for example, in silica microcapsules.

The concentration of MSM in a contemplated composition may be in a range of from about 0.1% to about 40% w/w. For example, from about 0.1% to about 3% w/w, from about 0.5% to about 2% w/w, from about 3% to about 5% w/w, from about 0.1% to about 5% w/w, from about 5% to about 10% w/w, from about 5% to about 7% w/w, from about 7% to about 10% w/w, from about 6% to about 8% w/w, from about 6% to about 9% w/w, from about 10% to about 20% w/w, from about 10% to about 15% w/w, from about 15% to about 20% w/w, from about 20% to about 40% from about 20% to about 30% w/w, or from about 30% to about 40% w/w, and any subranges and individual values therebetween.

In some embodiments the concentration of MSM is in a range of from about 0.10% to about 20% w/w, for example, from about 50% to about 10% w/w.

In some embodiments, strontium is provided to a contemplated composition as strontium salt, wherein the counter anion may be inorganic or organic counter anion. In some embodiment, strontium is in the form of a salt with a counter anion such as fluoride (F⁻), chloride (Cl⁻), bromide (Br⁻), iodide (I⁻) or nitrate.

In some embodiments, strontium is in the form of strontium chloride. In some embodiments the strontium is in the form of strontium chloride hexahydrate.

In some embodiments, strontium is in the form of strontium nitrate salt.

In some embodiments, strontium is in the form of an organic salt wherein the counter anion is an organic anion originating from, e.g., a carboxylic acid, an alkoxylate, an amino acid (especially, lysine, arginine, histidine, ornithine, aspartic acid, glutamic acid, proline, and cysteine), a peptide, a saturated or unsaturated organic acid, a saturated or unsaturated fatty acid.

In some embodiments, the organic counter anion is, for example, acetate, lactate, glycolate, tartrate, maleate, benzoate, propionate, salicylate, ascorbate, formate, succinate, folinate, aspartate, phthalate, oleate, palmitate, stearate, lauryl sulfate, lanolate, myristate, behenate, caseinate, cyclamate, pantothenate, EDTA or other polyaminopolycarboxylates, saccharin, thioglycolate, laurate, methylparaben, propylparaben, ricinoleate or sorbate anions.

In some embodiments, strontium is in the form of strontium acetate salt.

In some embodiments the concentration of elemental strontium in a disclosed composition is in a range of from about 0.1% to about 15% w/w. For example, from about 0.1% to about 2% w/w, from about 0.5% to about 1.5%, from about 2% to about 4% w/w, from about 2% to about 8% w/w, from about 4% to about 6% w/w, from about 5% to about 7% w/w, from about 6% to about 8% w/w, from about 7% to about 10% w/w, from about 8% to about 10% w/w, from about 9% to about 12% w/w, or from about 10% to about 15% w/w, and any subranges and individual values therebetween.

In some embodiments the concentration of strontium is in a range of from about 0.1% to about 10% w/w, for example, from about 2% to about 8% w/w.

The higher the concentration of BPO and/or azelaic acid, a higher concentration of the of strontium and/or MSM is recommended.

In some embodiments, a contemplated composition may comprise at least the following ingredients: (i) azelaic acid in an amount of form about 0.1% to about 40% w/w, form about 0.1% to about 5.0% w/w, form about 5.0% to about 15.0% w/w, or from about 10% to about 30% w/w; (ii) benzoyl peroxide in an amount of from 2.5% to about 10% w/w, form about 5% to about 10% w/w, form about 8% to about 15% w/w; (iii) one or more strontium salts in a total amount of from about 0.1% to about 10% w/w, or from about 2% to about 8% w/w; and (iv) MSM in an amount of from about 0.1% to about 20% w/w, or from about 5% to about 10% w/w.

Any of the compositions disclosed herein may further comprise at least one additional active ingredient (or active agent), such as, but not limited to, an alpha hydroxy acid (AHA), a beta hydroxy acid (BHA), a retinoid, an alpha keto acid, a dicarboxylic acid, arbutin, resorcinol, hydroquinone, kojic acid, myristic acid, sodium laureth sulfate, disodium laureth sulfosuccinate, sulfur, vitamin C a vitamin C derivative, or a cannabinoid.

In some embodiments, the additional active agent is, for example, an alpha hydroxy acid (AHA) such as glycolic acid, lactic acid, mandelic acid, tartaric acid, malic acid or citric acid; a beta hydroxy acid (BHA) such as salicylic acid or citric acid; a retinoid such as retinol, retinoic acid or any other derivative of vitamin A; an alpha keto acid such as pyruvic acid; arbutin such as alpha- or beta-arbutin; vitamin C and or a derivative thereof such as ascorbyl tetraisopalmitate or ascorbyl glucoside; sulfur; resorcinol, resorcinol monoacetate; hydroquinone; kojic acid; sodium laureth sulfate, disodium laureth sulfosuccinate; or medxtract Chamomile distilled.

The amount of any one or more of the additional active agents may be in the range of from 0.10% w/w to 70% w/w, for example, from about 2% to about 10% w/w, from about 5% to about 15% w/w, from about 12% to about 30% w/w, from about 25% to about 40% w/w, or from about 30% to about 50% w/w, depending on the type, duration and/or intended use of the formulation.

In some embodiments, the additional active ingredient is salicylic acid. In some embodiments, the additional active ingredient is glycolic acid. In some embodiments, the additional active ingredient is retinol and/or derivatives thereof.

A disclosed composition further comprises a dermatologically acceptable carrier. A “dermatologically acceptable carrier” as used herein means a carrier suitable for topical application to keratinous tissue, and compatible with the active ingredients in the formulation, that will not cause safety or toxicity concerns. Any of the dermatologically acceptable carriers well known in the art may be used in accordance with embodiments described herein in an amount of from about 0.1 to 99.10% w/w.

A disclosed composition may be formulated as a cosmetic product or as a medicament, i.e., a medicinal product, for treatment of, e.g., acne. Contemplated formulations may contain one or more dosage forms comprising benzoyl peroxide, azelaic acid, a strontium salt, and MSM. Essentially, at least one dosage form comprises BPO and azelaic acid and/or a derivative, salt or substitute thereof. When the formulation is intended for treatment of acne, it is termed herein an “anti-acne formulation”, and it may contain a combination of MSM and strontium, BPO and azelaic acid and, optionally, at least one additional anti-acne active agent and/or one or more anti-inflammatory active agents. For example, an anti-acne formulation may comprise azelaic acid, BPO, MSM, a strontium salt and one or more of lactic acid, sulfur, azeloglycine, resorcinol, and/or resorcinol monoacetate. Such formulations may be useful, e.g., for treating bumpy skin with papules and associated redness, helping to reduce discoloration. Exemplary formulations for treating acne are described in Example 1 herein.

The compositions disclosed herein present excellent physical and chemical stabilities, featured as intactness, texture stability, homogeneity and/or constant viscosity over time, for example, for at least 3 months, at temperatures ranging from 4 to 40° C.

Any of the compositions and formulations disclosed herein comprise, in addition to the relevant active agents and combination of MSM and strontium, further excipients, carriers and additives as well-known to a person skilled in the art. Such excipients include, for example, penetration enhancers, emulsifiers, humectants, solvents, surfactants, preservatives, moisturizers, fragrances, dyes/colorants, viscosity adjustment agents, emollients, binders, absorbents, buffering agents, chelating agents, conditioning agents, in various concentrations ranging from 0.010% to 70% w/w.

Disclosed compositions intended for topical application, usually comprise a penetration enhancer. Chemical permeation or penetration enhancers (CPEs) are molecules that interact with the constituents of skin's outermost and rate limiting layer stratum corneum (SC) and increase its permeability. A chemical penetration enhancement strategy is used in cosmetic and medicinal products disclosed herein in order to improve topical drug delivery. None-limiting examples of chemical penetration enhancers include ethanol, dimethyl sulfoxide, dimethyl isosorbide, fatty acid esters such (such as isopropyl myristate (IPM), propylene glycol monocaprylate (PGMC), propyleneglycolmonolaurate (PGML)), alkyl and benzoic acid esters (e.g., ethyl acetate, octyl salicylate (OS)), ether alcohols (e.g., Transcutol®), amides (e.g., azone (Laurocapram)), fatty acids (e.g., oleic acid (OA)), glycerin and glycols such as propylene glycol (PG).

In some embodiments, a contemplated cosmetic product or medicament may comprise propylene glycol as a penetration enhancer.

In cosmetic and skin care products disclosed herein propylene glycol may further be used as both a humectant and a conditioner. Propylene glycol acts as a humectant at a low concentration level: it secures the water and takes it to the outer layer of the skin. Hence, disclosed cosmetics products which have propylene glycol are also good for skin hydration and provide smoothness.

A disclosed composition may be formulated in the form of an ointment, a cream, a lotion, an oil, a solution (in some embodiments an aqueous solution), an emulsion, a nano-emulsion, a gel, a paste, a milk, an aerosol, a powder, or a foam. In some embodiments, the formulation is aqueous-based such as a gel or an aqueous solution. In some embodiments, the formulation is oil-in water emulsion, nano-emulsion, micro-emulsion, oil-in water cream, foam, lotion or spray.

In some embodiments, a contemplated composition is formulated as a cosmetic or skin care product and/or a medicament in the form of, e.g., a mask, a peel, a soap (liquid or solid), a shampoo, a shaving cream or gel, an after shave, a sunscreen, makeup and/or a makeup remover.

A disclosed composition formulated as a medicinal and/or cosmetic/skin care product enables using high concentrations of active medicinal and/or cosmetic ingredients at low pH without the typical irritations and side effects (redness etc.) resulting from the chemical irritant or the low pH. This is because the compositions described herein comprise MSM and a strontium salt which provide a shielding effect. Since both MSM and strontium provide protection to the skin, they are also termed herein “dermo shields”.

The combination of MSM and strontium may be used prior to, or post application of a combination of BPO and azelaic acid. When MSM and strontium are formulated in a dosage form without BPO and azelaic acid, they may be applied before or after application of the dosage form comprising BPO and azelaic acid, preferably in proximity thereto in order to have the desired effect of reducing or nulling irritation, erythema and/or neurogenic inflammation immediately.

The present disclosure provides more efficacious products for skin treatment, devoid of typical side effects such as redness, itching, burning, stinging, etc. The effectiveness of the products is increased because a subject is more likely to use these products as prescribed if there are no side effects or the side effects are mild. In addition, a subject applying, e.g., an anti-acne, anti-rosacea and/or anti-subboreal treatment is more likely to keep the products on for longer if the subject is not suffering or uncomfortable, thus benefiting the full effectiveness that is embodied in the product. In addition, disclosed products enable the subject or the practitioner to increase the amount of active ingredients since the side effects of, e.g., irritation and redness are eliminated or reduced, and effectiveness of the active ingredients is not harmed or compromised by the addition of strontium and MSM.

Typical modes of application of a disclosed cosmetic and/or therapeutic product include fingers, a physical applicator such as a brush, as stick, swab, tissue or cloth, or by applying or adhering a prepared applicator already containing the formulation such as a cloth mask.

### Methods of Treatment

In another aspect, the present disclosure relates to a method for treating, preventing, ameliorating, mitigating, and/or relieving a skin disease, disorder or conditions, which may benefit form topical co-administration of benzoyl peroxide (BPO) and azelaic acid, the method comprises administrating an effective amount of a contemplated composition and/or a contemplated formulation as disclosed herein.

In some embodiments, the skin disease, disorder or condition which may benefit form topical co-administration of BPO and azelaic acid is, for example, but not limited to, acne, rosacea, seborrhea, and/or demodicosis.

In yet a further aspect, the present disclosure relates to a method for treating, preventing, ameliorating, mitigating, relieving and/or reducing one or more of: neurogenic inflammation, stinging, itching, burning, redness, irritation, and/or other sensations and feelings associated with topical administration or application of benzoyl peroxide (BPO) and azelaic acid, the method comprises administrating an effective amount of a contemplated composition and/or a contemplated formulation as defined herein.

In some embodiments, BPO and azelaic acid are applied/administered simultaneously (co-applied or co-administered).

In some embodiments, BPO and/or azelaic acid are co-applied with a strontium salt and MSM for treating, preventing, and/or reducing neurogenic inflammation.

In some embodiments, the topical administration or co-administration of BPO and azelaic acid (together with MSM and strontium salt) is for treatment of, for example, but not limited to, acne, rosacea, seborrhea and/or demodicosis.

Acne is a common skin disease characterized by pimples on the face, chest, and back. It occurs when the pores of the skin become clogged with oil, dead skin cells, and bacteria.

Acne vulgaris, the medical term for common acne, is the most common skin disease. While acne can arise at any age, it usually begins at puberty and worsens during adolescence. Nearly 85% of people develop acne at some time between the ages of 12-25 years. Up to 20% of women develop mild acne.

The sebaceous glands (also called sebaceous follicles) lie just beneath the skin's surface. They produce an oil called sebum, the skin's natural moisturizer. These follicles open onto the skin through pores. At puberty, increased levels of androgens (male hormones) cause the glands to produce too much sebum. When excess sebum combines with dead, sticky skin cells, a hard plug or comedo, forms that blocks the pore. Mild noninflammatory acne consists of the two types of comedones, whiteheads and blackheads.

Moderate and severe inflammatory types of acne result after the plugged follicle is invaded by Propionibacterium acnes, a bacteria that normally lives on the skin. A pimple forms when the damaged follicle weakens and bursts open, releasing sebum, bacteria, and skin and white blood cells into the surrounding tissues. Inflamed pimples near the skin's surface are called papules; when deeper, they are called pustules. The most severe type of acne consists of cysts (closed sacs) and nodules (hard swellings). Scarring occurs when new skin cells are laid down to replace damaged cells.

State-of-the-art topical medications are available as cream, gel, lotion, or pad preparations of varying strengths. They include antibiotics such as erythromycin, clindamycin, and meclocycline (Meclan); comedolytics (agents that loosen hard plugs and open pores) such as the vitamin A acid (a vitamin A derivative, also known as retinoice acid, tretinoin and Retin-A), salicylic acid, adapalene (Differin), resorcinol, and sulfur. Drugs that act as both comedolytics and antibiotics, such as benzoyl peroxide, azelaic acid (Azelex), or benzoyl peroxide plus erythromycin (Benzamycin), are also used. These drugs may be used for months (at least 2 months) to years to achieve disease control.

Based on its oxidizing property, BPO presents sufficient antiseptic activity against Propionibacterium acnes bacteria, as well as antibiotic-resistant variants of P. acnes and Staphylococcus epidermidis that develop during long-term use of antimicrobials.

Rosacea is a skin disease typically appearing in people during their 30s and 40s. It is marked by redness (erythema) of the face, flushing of the skin, and the presence of hard pimples (papules) or pus-filled pimples (pustules), and small visible spider-like veins called telangiectasias, primarily in areas of the face, including the nose, cheeks, forehead, and chin, but sometimes also in the back, neck, scalp, arms and legs. In later stages of the disease, the face may swell, and the nose may take on a bulb-like appearance called rhinophyma.

Like acne, the skin can have pimples and papules. Unlike acne, however, people with rosacea do not have blackheads. In early stages of rosacea, patients typically experience repeated episodes of flushing. Later, areas of the face are persistently red, telangiectasia appear on the nose and cheeks, as inflamed papules and pustules. Overtime, the skin may take on a roughened, orange peel texture.

A topical agent applied directly to the face may be tried in addition to an oral antibiotic, or in its place for treating rosacea. Topical antibiotics are useful for controlling the papules and pustules of rosacea, but do not control the redness, flushing, and telangiectasias. Topical vitamin derivatives that are used in the treatment of acne also may have a role in the treatment of rosacea. Accumulating evidence suggests that topical application of isotretinoin and azelaic acid can reduce the redness and pimples. Some patients who use these medications experience skin irritation. For late stages of the disorder, a surgical procedure may be needed to improve the appearance of the skin. To remove the telangiectasias, a dermatologist may use an electrocautery device to destroy blood vessels.

Seborrhea is a disease of the sebaceous glands characterized by excessive secretion of sebum or an alteration in its quality, resulting in an oily coating, crusts, or greasy scales or cheesy plugs on the skin. It is generally associated with itching and/or burning.

Any of the methods disclosed herein is effective for therapeutic and/or cosmetic treatment of the skin, supports skin soothing, provides calmness of the skin during treatment and overall calmness of the skin, and/or provide a cosmetic effect to the skin such as improving skin appearance.

The combined application of strontium and MSM together with both BPO and azelaic acid, in accordance with embodiments disclosed herein, significantly extends the duration of the anti-irritation effect associated with BPO and/or azelaic acid topical administration as compared to absence of strontium and/or MSM co-application. The anti-irritant effect is immediate and in real-time, with no need to wait between the time of applying a contemplated composition and/or formulation and the time it starts to have an effect.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the present disclosure in a non-limiting fashion. Generally, the nomenclature used herein, and the laboratory procedures utilized in the present disclosure include molecular, chemical, biochemical and/or microbiological techniques. Such techniques are thoroughly explained in the literature. Other general references are provided throughout this document. The procedures therein are believed to be well known in the art and are provided for the convenience of the reader.

### Example 1

### Formulations for Treating Acne

Formulations for treating acne comprising BPO, azelaic acid, strontium salt, and MSM were prepared using the ingredients listed in Table 1:

TABLE 1
Main ingredients of an anti-acne formulation

| | Concentration | |
|---|---|---|
| | in final | |
| | product | |
| Ingredient | [weigh %] | Main Functions |
| Azelaic acid | 0.1-25 | anti-acne |
| Azeloglycine | 0.1-20 | anti-acne |
| (a derivative of azelaic acid) | | |
| Benzoyl peroxide (BPO) | 0.1-10 | anti-acne |
| MSM | 0.1-10 | dermo shielding |
| Strontium Chloride | 0.1-10 | dermo shielding |
| Glycerin | 3-10 | humectant, penetration |
| | | enhancer |
| Tween 20, polysorbate 20, | 0.1-2 | surfactants, emulsifiers |
| polysorbate 80 | | |
| AQUAXYL ™ | 2-3 | moisturizing, |
| (xylitylglucoside + | | stimulating the skin's |
| anhydroxylitol + xylitol) | | natural production of |
| | | hyaluronic acid |
| Propylene glycol | 1-10 | humectant, penetration |
| | | enhancer |
| Propanediol | 1-10 | viscosity controlling |
| Water | up to 100 | solvent |

Benzoyl peroxide was provided in the form of a stable nano-suspension (nano-emulsion), such as the commercially available Curoxyl™ BP 42 USP, an aqueous-based, micronized benzoyl peroxide dispersion (40%) that complies with the U.S. Food and Drug Administration's Benzoyl Peroxide Gel monograph. The BPO was added at the last stage of the formulation process at a temperature of 35° C.

Azelaic acid was provided as micronized particles, at least half of which were dissolved in propylene glycol and propanediol while heating, and half were added at the last stage of the formulation process. In this way, conglomerates formation, which results in a grainy cream, was prevented.

A contemplated composition formulated as anti-acne formulation may further comprise one or more of the following excipients: sequestering agents, antioxidants such as alpha-arbutin, fullerene, vitamin C, Q10, vitamin E; preserving agents; electrolytes; colorants; humectants; penetration enhancers such as propylene glycol, dimethyl isosorbite; moisturizers such as hyaluronic acid; essential oils that provide aroma (perfumes) such as Citrus medica limonum peel oil and limonene; active cosmetic agents such as vitamins (vitamin C, vitamin A (retinol), and/or salicylic acid; fatty acids such as myristic acid, stearic acid, oleic acid and/or palmitic acid; sphingolipids; silica; calming and/or protective agents.

Disclosed anti-acne formulations were carefully designed to incorporate the essential ingredients that would account for a desired anti-acne effect, featuring concentrations and relative amounts that would provide for a stable and highly effective products, formulated as creams or spreads.

Due to the keratolytic and anti-bacterial properties of BPO and azelaic acid, the formulations described herein are particularly useful for treating various forms of acne such as, but not limited to, acne rosacea, acne vulgaris, acne conglobate, cystic acne, acne comedones, severe nodulocystic acne, acne papulapostulosa, secondary acne such as solar acne and acne caused by drugs.

The disclosed formulations are further effective in treating sebaceous function disorders such as hyper seborrhea of acne or simple seborrhea, and/or seborrheic dermatitis.

### Example 2

### The Effect of Co-Application of MSM and Strontium in Treatment of Rosacea, Acne and Seborrhea with PBO and Azelaic Acid

Twenty subjects afflicted with rosacea, acne or seborrhea were provided with either a formulation disclosed in Example 1 comprising BPO, azelaic acid, strontium salt, and MSM (12 patients), a similar formulation but devoid of MSM and strontium (4 patients), which served as a control, or a placebo (4 patients).

All patients in the test group presented immediate improvement is their skin condition without adverse side effects, while about 90% of the control group presented serious and severe redness and irritation, which prevented any further use of the formulation.

These results clearly demonstrate that a combination of BPO and azelaic acid in topical administration is practically enabled for treating the above-mentioned skin diseases only upon co-administration of MSM and a strontium salt.

Furthermore, co-application of MSM and a strontium salt with either BPO or azelaic acid provided faster therapeutic results and reduced side effects as compared to application of BPO or azelaic acid, each alone, without MSM and strontium.

1. A composition comprising benzoyl peroxide, azelaic acid, a strontium salt, methylsulfonylmethane (MSM) and a dermatologically acceptable carrier, formulated as one or more dosage forms, wherein at least one dosage form comprises both benzoyl peroxide and azelaic acid.

2. The composition of claim 1, wherein the concentration of strontium is in a range of about 0.1% to 10% w/w and the concentration of MSM is in a range of from 0.1% to 20% w/w.

3. The composition of claim 1, wherein the concentration of benzoyl peroxide is in a range of from 0.1% to 30% w/w.

4. The composition of claim 3, wherein the concentration of benzoyl peroxide is at least one of: in the range of form about 2.5% to about 10% w/w, form about 5% to about 10% w/w, form about 8% to about 15% w/w, about 5% w/w or about 10% w/w.

5. The composition of claim 1, wherein the concentration of azelaic acid is in a range of from 0.1% to 40% w/w.

6. The composition of claim 5, wherein the concentration of azelaic acid is at least one of: in the range of form about 0.5% to about 25% w/w, form about 5% to about 30% w/w, form about 10% to about 25% w/w, form about 15% to about 35% w/w, about 15% w/w or about 25% w/w.

7. The composition of claim 1, wherein the benzoyl peroxide and/or azelaic acid are present as homogeneously distributed micronized particles.

8. The composition of claim 1, wherein the strontium salt is selected from the group consisting of strontium chloride, strontium acetate, strontium nitrate and strontium chloride hexahydrate.

9. The composition of claim 1, further comprising at least one additional active ingredient.

10. The composition of claim 9, wherein the at least one additional active ingredient is selected from the group consisting of an alpha hydroxy acid (AHA), a beta hydroxy acid (BHA), a retinoid, an alpha keto acid, a dicarboxylic acid, arbutin, resorcinol, hydroquinone, kojic acid, myristic acid, sodium laureth sulfate, disodium laureth sulfosuccinate, sulfur, vitamin C, a vitamin C derivative, a cannabinoid, an azelaic acid derivative, salt and/or a prodrug, diaryl peroxide, alkyl aryl peroxide, and/or a cycloalkyl aryl peroxide.

11. A cosmetic product or medicament comprising a composition according to claim 1.

12. The cosmetic product or medicament of claim 11, comprising one or more ingredients selected from antioxidants, preserving agents, electrolytes, colorants, humectants, penetration enhancers, moisturizers essential oils, active cosmetic agents, vitamins, essential fatty acids, perfumes, or calming and protective agents.

13. The cosmetic product or medicament of claim 11, comprising propylene glycol as a penetration enhancer.

14. The cosmetic product or medicament of claim 11, formulated as at least one of: an ointment, a cream, a spread, a lotion, an oil, a solution, an emulsion, a nano-emulsion, a gel, a paste, a milk, an aerosol, a powder, or a foam.

15. The cosmetic product or medicament of claim 11, which is physically and/or chemically stable for a period of at least 3 months under 4 to 40° C.

16. A method for treating, preventing, ameliorating, and/or relieving a skin disease, disorder or conditions which may benefit form topical co-administration of benzoyl peroxide (BPO) and azelaic acid, comprising administrating to a subject in need thereof an effective amount of a composition according to claim 1 or a cosmetic product or medicament comprising same.

17. The method of claim 16, wherein the skin disease, disorder or condition is at least one of acne, rosacea or seborrhea.

18-20. (canceled)

21. The cosmetic product or medicament according to claim 11, wherein the concentration of strontium is in a range of about 0.1% to 10% w/w, the concentration of MSM is in a range of from 0.1% to 20% w/w, concentration of benzoyl peroxide is in a range of from 0.1% to 30% w/w and the concentration of azelaic acid is in a range of from 0.1% to 40% w/w.

22. The cosmetic product or medicament according to claim 11, wherein the benzoyl peroxide and/or azelaic acid are present as homogeneously distributed micronized particles
